(19) United States
(12) Patent Application Publication
(10) Pub. No.: US 2010/0038280 A1
(43) Pub. Date: Feb. 18, 2010

(54) Hermetically Sealed Package for A Therapeutic Diffusion Device

(76) Inventors: Amie B. Franklin, Mill Valley, CA (US); Olivier B. Postel, Redwood City, CA (US)

Correspondence Address: CENTRAL COAST PATENT AGENCY, INC, 3 HANGAR WAY SUITE D, WATSONVILLE, CA, 95076, US

(21) Appl. No.: 12/208,709

(22) Filed: Sep. 11, 2008

Related U.S. Application Data
(60) Provisional application No. 61/089,374, filed on Aug. 15, 2008.

(51) Int. Cl. A61B 19/02; B32B 37/06

(52) U.S. Cl. 206/572; 156/60

(57) ABSTRACT

A package for a diffusion device including more than one device layer and at least one reservoir containing a therapeutic agent, the package including at least one packaging layer, and at least one sealing surface disposed along one or more edges of the at least one packaging layer. The package is characterized in that the sealing surface engages one of a device layer or another packaging layer to form the package.

### CROSS-REFERENCE TO RELATED APPLICATIONS

The present application claims priority to U.S. provisional patent application Ser. No. 61/089,374 filed on Aug. 15, 2008, entitled “Method and Apparatus for Packaging a Diffusion Device,” which is included herein at least by reference.

### BACKGROUND OF THE INVENTION

1. Field of the Invention

The present invention is in the field medical technologies and more particularly the treatment of tissue via a diffusion type device, the invention pertaining to packaging of such devices for transport and storage before use.

2. Discussion of the State of the Art

In the medical field it is often required to treat tissue of patients in a manner which requires an extended period of bandaging or covering with medication diffusing patches or like devices that work to keep tissue moist, promote healing, to prevent maceration, possible infection, or to prevent any jolting or other physical disruption of a particular tissue area.

One example of such a therapeutic device is a multilayered diffusion dressing or device typically applied over a wound or graft to promote new skin growth in an area where the original skin was lost or removed by surgery. Such devices have different names in the art but may be technically referred to as therapeutic diffusion devices because of an active delivery (diffusion) of some therapeutic agent, often delivered in the form of a gas through an interfacing diffusion layer of the dressing or device to the affected tissue over time.

Such devices may vary architecturally according to therapeutic need. For example the therapeutic agent may be a gas, a salve, an antibiotic, a growth hormone, or a mixture of agents. In many cases a medium is also present in the device to help contain and deliver the agent.

One problem with such diffusion devices is that, unless contained, they have no shelf life once created from the materials and therapeutic substances required to promote recovery in particular tissue conditions. The products are often built for the purpose at the time they are needed. Currently tissue-targeted therapeutic diffusion devices are available off-the-shelf that can be safely stored until such time that the device may be required for use on a patient. However, one limitation of these devices known to the inventor is that they must be kept in a pouch or similar container and removed from the pouch before use.

Therefore, what is clearly needed is a hermetically sealed package for diffusion devices and methods of fabrication thereof to preserve the integrity of the therapeutic agents and mediums within the device for storage and transport before needed in the field.

### SUMMARY OF THE INVENTION

The problem stated above is that for a diffusion dressing, there are no reliable means for preserving therapeutic agents from the time of manufacture to the time of use of the products to treat tissue aside from containment of the dressing in a pouch or similar container. Many patients have diffusion bandages that wear out, become unsealed, and are not reliably refillable with new agent during a course of treatment. The inventors therefore considered functional elements of a diffusion dressing, looking for elements that exhibit impermeable and permeable properties that could potentially be harnessed to provide shelf life for stored therapeutic agents and breathability in some cases for tissue dressings but in a manner that would not present problems or hardships for users.

Every diffusion dressing is charged with some therapeutic agent that is expelled from the device through a diffusion process one effect there being that the dressing eventually becomes depleted of agent and is no longer effective. Most such diffusion devices employ gasses infused into a reservoir in the device, the gases being diffused into a tissue area through a special diffusion layer of the device. Diffusion layers and adhesive layers for application of the dressing over a wound are typically a part of such apparatus.

The present inventors realized in inventive contemplation that if, at the point of manufacture, therapeutic gasses could be preserved within a diffusion dressing for an extended period of time, significant convenience might result for practitioners and patients. The inventor therefore constructed a unique packaging arrangement for diffusion devices that allowed gases to be stored within such devices for an indeterminate period and that allowed the packaged devices to be activated to diffuse the gasses at the appropriate time when sealing the device over the tissue to be treated. A significant improvement in therapeutic benefit resulted with no impediment to practitioners or patients use of such diffusing devices created.

Accordingly, in an embodiment of the present invention, a method is provided for packaging a diffusion device for treating tissue, the device including more than one device layer and at least one reservoir containing a therapeutic agent comprising the steps (a) positioning the diffusion device over top of a first packaging layer; (b) positioning a second packaging layer over top of the diffusion device and the first packaging layer; and (c) forging a seal, the seal incorporating at least the first packaging layer and the second packaging layer.

In one aspect of the method the first and second packaging layers are one of Polyvinylidene Chloride or Polyurethane. In one aspect in step (a) the first packaging layer has a peripherally disposed sealing surface and in step (b) the second packaging layer has a peripherally disposed sealing surface. In one aspect in step (c) the seal is forged using heat and pressing the layers together. In one aspect variation in step (c) the seal includes a device layer and the device layer is an adhesive layer. In another aspect variation, in step (c) the seal is an adhesive bead applied to the peripheral edges of the first and second packaging layers. In a preferred aspect the seal is a hermetic seal.

According to another aspect of the present invention, a method is provided for packaging a diffusion device for treating tissue, the device including more than one device layer and at least one reservoir containing a therapeutic agent comprising the steps (a) positioning the diffusion device over top of a packaging layer, and (b) forging a seal between the packaging layer and a device layer. In one aspect of this method, the device layer is an adhesive layer. In one aspect in step (b) the seal is forged using heat and pressing the layers together. In one aspect of the method, the packaging layer is one of Polyvinylidene Chloride or Polyurethane. In one aspect in step (b) the seal is an adhesive bead running peripherally about the device.

According to another aspect of the invention, a method is provided for packaging a diffusion device for treating tissue, the device including more than one device layer and at least one reservoir containing a therapeutic agent comprising the steps (a) positioning the diffusion device over top of a first packaging layer, (b) placing a second packaging layer over the uppermost device layer of the device, (c) forging a seal between the second packaging layer and the uppermost device layer, and (d) forging a seal between the first packaging layer and an intermediate device layer.

In one aspect of this method, in step (c) the second packaging layer is not peeled off until sometime after applying the device to an area of tissue to allow the device to breathe. In one aspect of the method in step (d) the intermediate device layer is an adhesive layer. Also in one aspect the first and second packaging layers are Polyvinylidene Chloride or Polyurethane.

In another aspect of the invention, a package is provided for a diffusion device, the device including more than one device layer and at least one reservoir containing a therapeutic agent. The package includes at least one packaging layer, and at least one sealing surface disposed along one or more edges of the packaging layer. The packaged is characterized in that the sealing surface engages one of a device layer or another packaging layer to form the package.

In one embodiment the at least one packaging layer is a Polyvinylidene Chloride or a Polyurethane and the sealing surface is activated by heat treatment. In one embodiment the package includes two packaging layers incorporated independently of one another while enclosing a diffusion device. In one embodiment a packaging layer includes one or more sub-layers that are removable.

### BRIEF DESCRIPTION OF THE DRAWING FIGURES

FIG. 1 is an elevation view of a packaged diffusion device according to an embodiment of the present invention.

FIG. 2A is a partial view of a packaged diffusion device with a peripheral multi-layered seal.

FIG. 2B is a partial view of a packaged diffusion device with a peripheral bead seal.

FIG. 3 is an elevation view of a diffusion device packaged according to an alternate embodiment.

FIG. 4A is a partial view of a packaged diffusion device with a multi-layered seal.

FIG. 4B is a partial view of a packaged diffusion device with a peripheral bead seal.

FIG. 4C is a partial view of a packaged diffusion device with the seal located more inward and away from the peripheral edge if the device.

FIG. 5 is an elevation view of a package diffusion device according to a further embodiment of the invention.

### DETAILED DESCRIPTION

The inventors provide a method for containing a therapeutic agent, for example a gas, in a diffusion device. The method enabled by apparatus also protects such devices from leaking therapeutic agents or mediums and prevents specific outside elements from entering the volume or volumes of such devices and diluting or corrupting therapeutic agents.

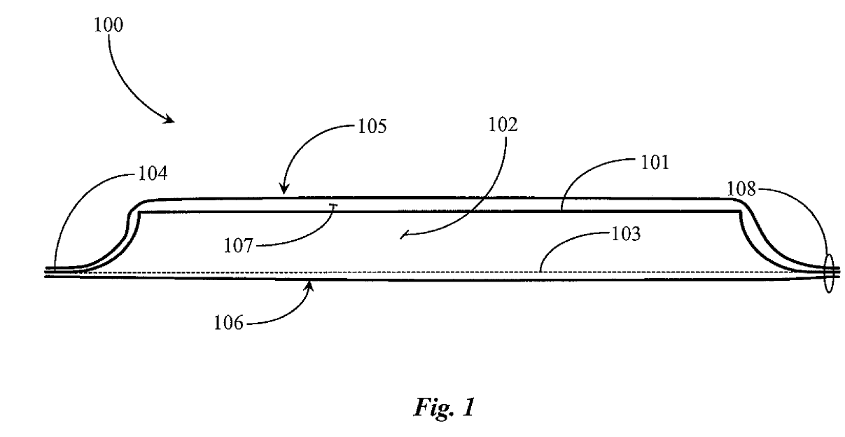

FIG. 1 is an elevation view of a packaged diffusion device 100 according to an embodiment of the present invention. diffusion device 100 is typical of a diffusion dressing known to the inventor and includes a diffusion layer 103 through which a therapeutic agent stored in volume 102, for example, diffuses into a tissue wound or other targeted tissue. Device 100 includes an occlusive layer 101 that is impermeable to the therapeutic agent in a preferred embodiment. An adhesive layer 104 may be provided peripherally around the diffusion device for enabling device adhesion to tissue to be treated. In a preferred embodiment, the seal is hermetically applied.

In this example device 100 has a finite shape with predictable dimensions. In one embodiment however, device 100 may be cut from a roll leaving the longitudinal dimension variable. Occlusive layer 101 may, in one embodiment, be somewhat permeable to the therapeutic agent stored in volume 102 without departing from the spirit or scope of the present invention.

The inventor provides packaging to device 100. Without packaging device 100 may typically be filled with gel, emulsion, gas or any other medium containing therapeutic agent, or ambient air at the time of need of the agent. Diffusion layer 103 begins diffusing therapeutic agent immediately after charging the device.

Diffusion device 100 is packaged in this embodiment by placing the device between two separate packaging layers. A top packing layer 105 is provided to cover the top area of diffusion device 100. A bottom packing layer 106 is provided to cover the bottom area of device 100. Layers 105 and 106 may be manufactured of the same material, preferably impermeable to therapeutic agents and delivery mediums, as well as the outside air.

Layers 105 and 106 may be oriented such that they are symmetrically opposed during packaging with the diffusion device positioned there between. Layers 105 and 106 may be manufactured of Polyvinylidene chloride or other poly-based materials. Other materials that are impermeable to therapeutic agents and mediums used in the diffusion device may include certain metallic based materials, other inorganic materials or a combination of these. It is desired that the material be flexible and sealable to other typical device layers.

In this example a hermetic seal 108 is applied about the periphery of package 100 and involves top packing layer 105, adhesive layer 104, and lower packing layer 106. Adhesive layer 104 may incorporate layers 101 and 103 without packaging. Seal 108 prevents escape of any therapeutic agent or medium from anywhere within the package. Volume 102 may be filled or charged with therapeutic agent at the time of packaging, preferably after a seal 108 is substantially complete. This is because gas will immediately diffuse through diffusion layer 103 when the device is charged. In one embodiment a one-way valve stem (not illustrated) is provided and adapted to accept compressed agents into one or more reservoirs of the diffusion device. In that case the packaging incorporates the valve stem in its design.

A packaging volume 107 is created by virtue of the sealing of layers 105 and 106 about the diffusion device. Volume 107 may be filled with the same therapeutic agent that the diffusion device is filled with and at a specific pressure to equalize the pressure within the device and the package effective preventing diffusion of the internal therapeutic agent from within the device into the packaging. In this way, device 100 may be stored in a charged state and used at a time when it is needed. Packaging layer 106 may be removed just before device application to tissue thereby activating the diffusion of the device. In another embodiment, the device may be charged and recharged with therapeutic agent as needed while being stored or while in use treating tissue.

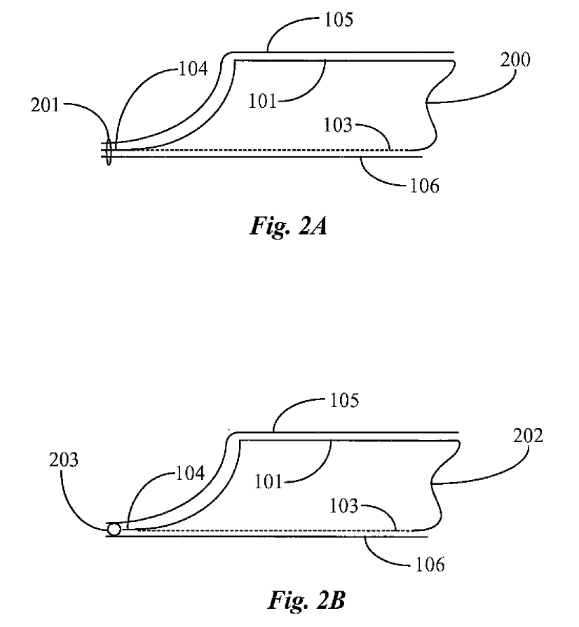

FIG. 2A is a partial view of a packaged diffusion device 200 with a peripheral multi-layered seal. FIG. 2B is a partial view of a packaged diffusion device 202 with a peripheral bead seal. Referring now to FIG. 2A, device 200 is analogous to device 100 described above with respect to FIG. 1. Diffusion layer 103 and occlusive layer 101 define the diffusion device. Adhesive layer 104 is also apparent on device 200.

Device 200 may be sealed to packaging layers 105 and 106 by a hermetic seal 201 that incorporates the multiple layers into the seal. Application of adhesive to all of the layer surfaces may be helpful. The seal formed is hermetic and does not allow gas to escape the device nor air to enter the device.

Referring now to FIG. 2B, device 202 is provided and illustrated. The configuration of device 2B is largely the same as that for device 200. Many of the parts are the same. Top occlusive layer 105 and bottom layer 106 are common to both devices and comprise the packaging in both instances. In this case, device 202 has a peripheral bead seal 203 disposed about the device and packaging instead of a multi-layered sealing approach described above. Bead seal 203 adheres to top packaging layer 105 and to bottom packaging layer 106. Bead 203 may also incorporate the area just along the outer edge of the adhesive layer. In the case of a bead seal, the act of removing the packaging layers may cause the seal to dislodge from any part of diffusion device 100 thereby ensuring the integrity adhesive layer 104 used to seal the device to an area of tissue to be treated.

One with skill in the art of forming seals will recognize there are several differing methods available for sealing packaging layers 105 and 106 of device 202. Press seal or heat seal techniques may be used. In mass production, a machine may be adapted to package the diffusion device including sealing the package. As described further above, the manufacture and packaging of the diffusion device may be undertaken relative to separate finite packages or to a packaged roll which contains the diffusion devices separable such as by perforation or the like.

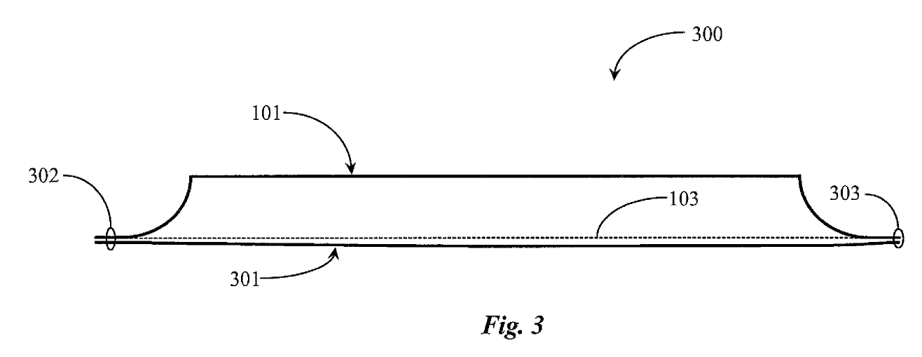

FIG. 3 is an elevation view of a diffusion device 300 packaged according to an alternate embodiment. Device 300 is very similar to device 100 described further above. An occlusive layer 101 and diffusion layer 103 define the diffusion device along with an adhesive layer 104. In this embodiment, layer 101 is completely impermeable to any transfer of therapeutic agent or medium and therefore functions as part and parcel of the packaging to prevent escape of agent from the device. Layer 101 in this case is also impermeable to the ambient air and other environmental chemistries like dew, moisture, etc.

In this embodiment only one containment or packaging layer 301 is provided. Layer 301 resides directly adjacent to and underneath diffusion layer 103. Layer 301 may be sealed directly to adhesive layer 104 using a multi-layer adhesion where all contact surfaces have adhesive applied forming seal 302, or a bead sealing technique resulting in a bead seal 303.

In this embodiment the volume between layer 301 and diffusion layer 103 may be filled with the same therapeutic agent as is contained within the device. Also in this embodiment a user would remove layer 301 such as by peeling it off of the diffusion device when ready to use the device. Charging the volume between the packaging layer 301 and the diffusion layer of device 300 may aid to equalize pressure slowing or halting diffusion until the layer is removed.

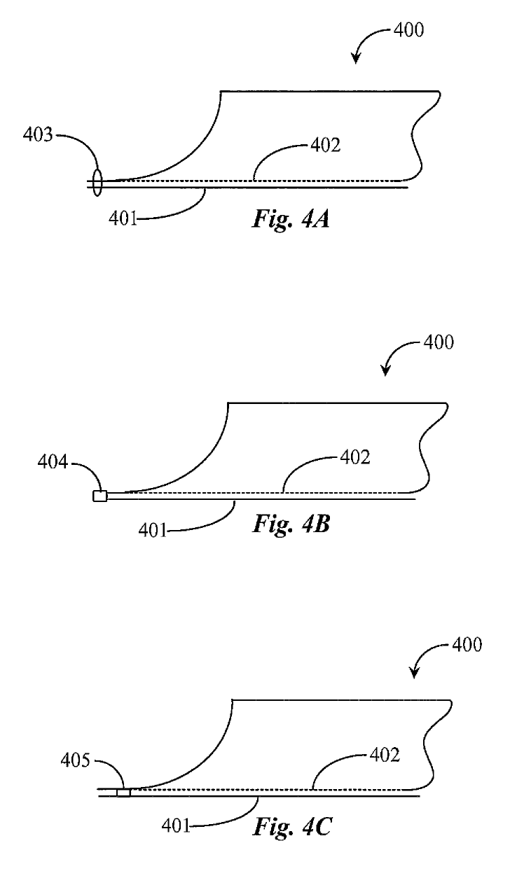

FIG. 4A is a partial view of a packaged diffusion device with a multi-layered seal. FIG. 4B is a partial view of a packaged diffusion device with a peripheral bead seal. FIG. 4C is a partial view of a packaged diffusion device with the seal located more inward and away from the peripheral edge if the device. Referring now to FIG. 4A, a multi-layered seal 403 is applied between diffusion layer 402 and containment or packaging layer 401 of device 400. This embodiment is analogous to device 300 and seal 302 of FIG. 3. Packaging layer 401 would be removed before use and an adhesive layer analogous to adhesive layer 104 would secure the device to the targeted tissue area.

Referring now to FIG. 4B, layer 401 is sealed to device 400 using an adhesive bead 404 applied peripherally about the device. Referring now to FIG. 4C, an adhesive bead seal 405 is applied further into the device. This latter case is significant if the adhesive section of device 400 has multiple sections with one of them being permeable to the therapeutic agent.

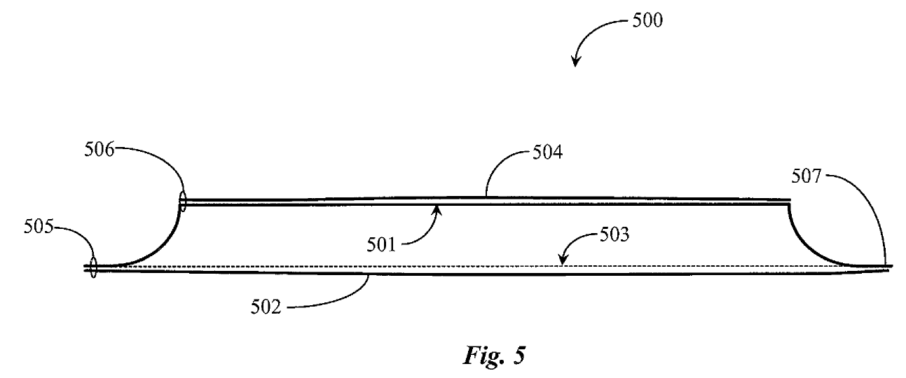

FIG. 5 is an elevation view of a package diffusion device 500 according to a further embodiment of the invention. Device 500 includes an upper layer 501 that may not be absolutely impermeable to the therapeutic agent. Device 500 includes a diffusion layer 503. Packaging in this case comprises a lower packaging layer 502 and an upper packaging layer 504.

Packaging layer 504 is attached to layer 501 using common film adhesive techniques known to and available to the inventor. In this example layer 504 is sealed to layer 501 with a peripheral seal 506 to provide a hermetic seal. Layer 504 is adapted to be removed after diffusion device 500 is in use on a tissue area to allow the device (dressing) to breathe through the somewhat permeable layer 501.

Packaging layer 502 is adapted to be sealed to device 500 adjacent to and beneath diffusion layer 503 using a peripheral seal 505 different types of which have been described further above. Packaging layer 502 should be impermeable to a therapeutic agent contained in device 500. Packaging layer 504 should also be impermeable to the therapeutic agent. For storage purposes it is also assumed at least that the sides of the package are also impermeable to the therapeutic agent thereby justifying the use of packaging layer 504 to “create” impermeability to a therapeutic agent stored within the device.

In one embodiment, layer 504 covers the entire upper surface of the whole device including the adhesive layer disposed peripherally about the device. In one embodiment which may include the embodiment just mentioned, layer 504 may include more than one sub-layer which may be independently peeled away from the covering layer. Such a sub-layer may be crafted to slightly overlap a preceding sub-layer to enable easy peel-off of the sub-layer. Other layers may also comprise removable sub-layers without departing from the spirit and scope of the invention such as packaging layer 502 of this example and diffusion layer 503 of this example.

In one embodiment layer 501 is a vapor transfer barrier to allow tissue to dry and to prevent wound maceration and is in a preferred embodiment mostly impermeable to the therapeutic agent contained in the device. Layer 502 is impermeable to the therapeutic gas and to the outside air and can be made from materials such as Polyvinylidene Chloride, or Polyurethane.

It will be apparent to one with skill in the art that the packaging of the invention may be provided using some or all of the mentioned features and components without departing from the spirit and scope of the present invention. It will also be apparent to the skilled artisan that the embodiments described above are exemplary of inventions that may have far greater scope than any of the singular descriptions. There may be many alterations made in the descriptions without departing from the spirit and scope of the present invention.

What is claimed is:

1. A method for packaging a diffusion device for treating tissue, the device including more than one device layer and at least one reservoir containing a therapeutic agent comprising the steps:
(a) positioning the diffusion device over top of a first packaging layer;
(b) positioning a second packaging layer over top of the diffusion device and the first packaging layer; and
(c) forging a seal, the seal incorporating at least the first packaging layer and the second packaging layer.

2. The method of claim 1 wherein the first and second packaging layers are one of Polyvinylidene Chloride or Polyurethane.

3. The method of claim 1 wherein in step (a) the first packaging layer has a peripherally disposed sealing surface.

4. The method of claim 1 wherein in step (b) the second packaging layer has a peripherally disposed sealing surface.

5. The method of claim 1 wherein in step (c) the seal is forged using heat and pressing the layers together.

6. The method of claim 1 wherein in step (c) the seal includes a device layer and the device layer is an adhesive layer.

7. The method of claim 1 wherein in step (c) the seal is an adhesive bead applied to the peripheral edges of the first and second packaging layers.

8. The method of claim 1 wherein in step (c) the seal is a hermetic seal.

9. A method for packaging a diffusion device for treating tissue, the device including more than one device layer and at least one reservoir containing a therapeutic agent comprising the steps:
(a) positioning the diffusion device over top of a packaging layer; and
(b) forging a seal between the packaging layer and a device layer.

10. The method of claim 9 wherein in step (b) the device layer is an adhesive layer.

11. The method of claim 9 wherein in step (b) the seal is forged using heat and pressing the layers together.

12. The method of claim 9 wherein the packaging layer is one of Polyvinylidene Chloride or Polyurethane.

13. The method of claim 9 wherein in step (b) the seal is an adhesive bead running peripherally about the device.

14. A method for packaging a diffusion device for treating tissue, the device including more than one device layer and at least one reservoir containing a therapeutic agent comprising the steps:
(a) positioning the diffusion device over top of a first packaging layer;
(b) placing a second packaging layer over the uppermost device layer of the device;
(c) forging a seal between the second packaging layer and the uppermost device layer; and
(d) forging a seal between the first packaging layer and an intermediate device layer.

15. The method of claim 14 wherein in step (c) the second packaging layer is not peeled off until sometime after applying the device to an area of tissue to allow the device to breathe.

16. The method of claim 14 wherein in step (d) the intermediate device layer is an adhesive layer.

17. The method of claim 14 where in the first and second packaging layers are Polyvinylidene Chloride or Polyurethane.

18. A package for a diffusion device including more than one device layer and at least one reservoir containing a therapeutic agent comprising:
at least one packaging layer; and
at least one sealing surface disposed along one or more edges of the packaging layer;
characterized in that the sealing surface engages one of a device layer or another packaging layer to form the package.

19. The package of claim 18 wherein the at least one packaging layer is a Polyvinylidene Chloride or a Polyurethane and the sealing surface is activated by heat treatment

20. The package of claim 18 including two packaging layers incorporated independently of one another while enclosing a diffusion device.

21. The package of claim 18 wherein a packaging layer includes one or more sub-layers that are removable.

22. The method of claim 14 wherein in step (b) the second packaging layer includes at least one sub-layer that is removable.
